(19) United States
(12) Patent Application Publication
(10) Pub. No.: US 2026/0026977 A1
(43) Pub. Date: Jan. 29, 2026

(54) DRESSING

(71) Applicant: Aatru Medical, LLC, Cleveland, OH (US)

(72) Inventor: Thomas E. Lash, Chardon, OH (US)

(21) Appl. No.: 18/782,152

(22) Filed: Jul. 24, 2024

(51) Int. Cl. A61F 13/05; A61B 46/20

(52) U.S. Cl. CPC A61F 13/05; A61B 46/20; A61B 2046/205

(57) ABSTRACT

A dressing assembly includes a drape, pressure-sensitive adhesive, a gasket, a gasket release liner and a border release liner. The drape is formed of a flexible material capable of maintaining a negative pressure underneath the drape upon application of a vacuum. The pressure-sensitive adhesive is provided on a skin-facing surface of the drape. The gasket is secured on the skin-facing surface and has a frame shape with an inner edge defining an opening and an outer edge offset inwardly from an outer periphery of the drape leaving a border of pressure-sensitive adhesive around the gasket. The gasket release liner is in contact with the gasket. The border release liner is in contact with the border of pressure-sensitive adhesive around the gasket. The gasket release liner is removable from the gasket to expose a lower surface of the gasket without removing the border release liner from the border of pressure-sensitive adhesive.

### BACKGROUND

Negative pressure is a term used to describe a pressure that is below normal atmospheric pressure. U.S. Pat. No. 11,007,083 B2 describes a known dressing for use with negative pressure wound therapy (NPWT). Relatively larger medical dressings, particularly those designed for joint areas like knees, often face challenges in application over a wound, surgical incision, or other tissue site. Traditional dressings may not adequately conform to the contours of joints, leading to poor adhesion or air leakage, which can be detrimental for NPWT.

### SUMMARY

In view of the foregoing, a dressing assembly is provided that includes a drape, pressure-sensitive adhesive, a gasket, a gasket release liner and a border release liner. The drape is formed of a flexible material capable of maintaining a negative pressure underneath the drape upon application of a vacuum. The pressure-sensitive adhesive is provided on a skin-facing surface of the drape. The gasket is secured on the skin-facing surface of the drape and has a frame shape with an inner edge defining an opening and an outer edge offset inwardly from an outer periphery of the drape leaving a border of pressure-sensitive adhesive around the gasket. The gasket release liner is in contact with the gasket. The border release liner is in contact with at least a portion of the border of pressure-sensitive adhesive around the gasket. The gasket release liner is removable from the gasket to expose a lower surface of the gasket without removing the border release liner from the border of pressure-sensitive adhesive around the gasket.

A method of assembling a dressing assembly can include affixing an absorbent pad to a skin-facing surface of the drape, affixing a gasket on the skin-facing surface of the drape surrounding the absorbent pad leaving a border of adhesive-coated drape around the gasket, covering the border of adhesive-coated drape around the gasket with a border release liner, and covering a lower surface of the gasket with a gasket release liner.

A method of applying a dressing to skin can include removing a gasket release liner from a lower surface of a gasket that is affixed to a drape, positioning the gasket over a tissue site and bringing the gasket in contact with the skin, removing a border release liner from a border of adhesive-coated drape around the gasket with the gasket positioned over the tissue site, and bringing the border of adhesive-coated drape around the gasket in contact with the skin.

### BRIEF DESCRIPTION OF THE DRAWINGS

FIG. 1 is a schematic top plan view of an example of a dressing.

FIG. 2 is an exploded side view of the dressing depicted in FIG. 1.

FIG. 3. is a schematic top plan view of a dressing assembly including the dressing of FIG. 1 with a gasket release liner and a border release liner.

FIG. 4. is a schematic top plan view of another dressing assembly including the dressing of FIG. 1 with a gasket release liner and two border release liners.

FIG. 5 is a schematic top plan view of another dressing assembly including the dressing of FIG. 1 with a gasket release liner and multiple border release liners.

FIG. 6 is a flow diagram depicting an example of a method of assembling the dressing assemblies depicted in FIGS. 3, 4 or 5.

FIG. 7 is a flow diagram depicting an example of a method of applying the dressing depicted in FIGS. 3, 4 or 5.

### DETAILED DESCRIPTION

The invention is not limited in its application to the details of construction and arrangement of components provided in the following description or illustrated in the attached drawings. The invention is capable of other embodiments and being practiced in various manners. The phraseology and terminology used herein is for the purpose of description and should not be regarded as limiting. Moreover, the use of “including,” “comprising,” or “having” and variations thereof is meant to encompass the items listed thereafter and equivalents thereof as well as additional items.

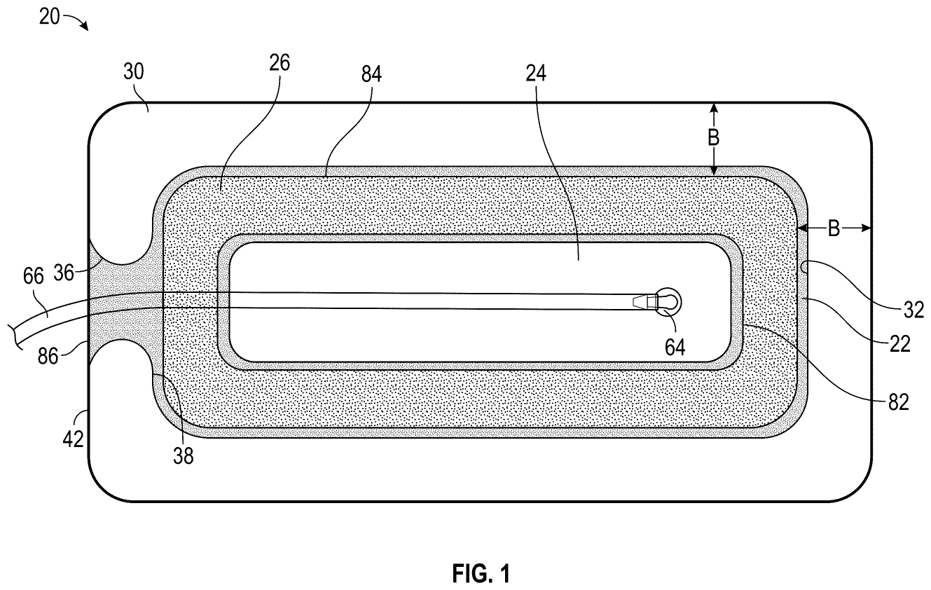

The present disclosure generally relates to negative pressure-type wound dressings, but the dressings described herein need not always be used with negative pressure. FIG. 1 depicts an embodiment of a dressing 20 that is useful to create a pressure that is below normal atmospheric pressure beneath the dressing 20. The dressing 20 generally includes a drape 22, an absorbent pad 24, and a gasket 26. The dressing 20 is provided with multiple release liners, which will be described in more detail below, to form a dressing assembly. Even though the dressing 20 is shown to be rectangular in shape in plan view, the dressing 20 could take other shapes, such as oval shaped, or another shape. The arrangement of the components of the dressing 20 and the material from which the components are constructed facilitates conformity of the dressing 20 around a wound, surgical incision, or other tissue site (hereinafter simply referred to as a “tissue site”) so as to maintain a negative pressure environment beneath the drape 22 and around the tissue site for extended periods of time, and also allows easier handling for placement of the dressing 20 onto the skin.

The drape 22 is a thin film capable of maintaining a negative pressure underneath the drape 22 upon application of a vacuum. The thin film from which the drape 22 is made can be substantially impermeable to liquids but somewhat permeable to water vapor, while still being capable of maintaining negative pressure underneath the drape. For example, the thin film material from which the drape 22 is made may be constructed of polyethylene, polyurethane or other semi-permeable material such as that sold under the Tegaderm® brand or 9834 TPU tape available from 3M. Similar films are also available from other manufacturers. Even though the film from which the drape 22 is made may transmit water vapor, these films are still capable of maintaining negative pressure underneath the drape 22 when an appropriate seal is made around the periphery of a tissue site. Even though the drape 22 is shown to be rectangular in shape in plan view, the drape 22 could take other shapes.

The drape 22 can be cast onto a casting sheet 30, which can be made from paper or plastic. When the dressing 20 is assembled, the casting sheet 30 can be kiss cut to provide a casting sheet opening 32. The drape 22 can be made from a transparent material such that the absorbent pad 24 and the gasket 26 can be visible within a “window” defined by the casting sheet opening 32 in the casting sheet 30. A cut out 36 can extend from an internal edge 38 of the casting sheet 30, after the material that defines the casting sheet opening 32 has been removed, to a peripheral edge 42 of the casting sheet 30. Since the drape 22 is made from a very thin film, by providing a frame-like structure that is the casting sheet 30 connected with the drape 22, application of the dressing 20 is facilitated by way of providing a relatively stiffer or more rigid casting sheet 30 to grasp while placing the dressing 20 over the tissue site. After the dressing 20 has been placed, the casting sheet 30 can be grasped at the cut out 36 and pulled towards a corner to remove the casting sheet 30 from the drape 22. As is evident in the embodiment depicted in FIG. 1, the casting sheet 30 is kiss cut around the area of the gasket 26 so as to allow for the person placing the dressing 20 onto the tissue site to see both the absorbent pad 24 and the gasket 26 during placement of the dressing 20.

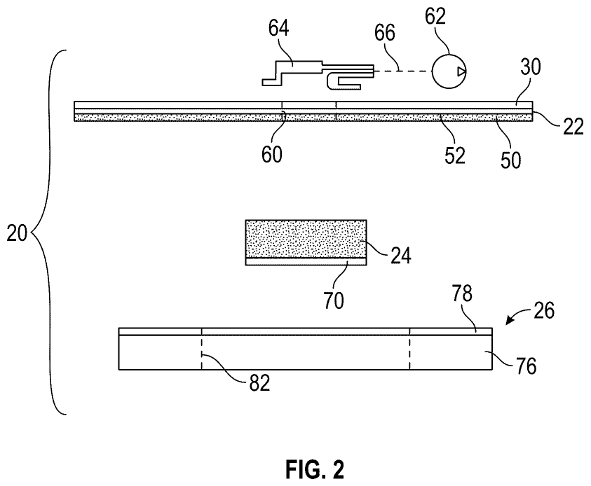

With reference to FIG. 2, a pressure-sensitive adhesive 50, which can be acrylic-based, is applied on a skin-facing surface 52 of the drape 22. Other types of adhesives could be applied to the drape 22, however, a pressure-sensitive acrylic-based adhesive is known to provide strong initial tack that can last for a relatively long time, for example a few days, when in contact with the skin. The pressure-sensitive adhesive 50 can be applied over an entirety of the skin-facing surface 52 of the drape 22, which can also be useful to retain other components of the dressing 20 during assembly.

The drape 22 can also include a hole 60 (see FIG. 2), which can allow for the connection of a vacuum source 62 (schematically depicted in FIG. 2) to the dressing 20. The hole 60 can be cut through the casting sheet 30 (prior to removal of the portion of the casting sheet 30 which forms the casting sheet opening 32) and the drape 22 within an area surrounded by the gasket 26. A fitting 64 can be placed over the hole 60 and connect to the vacuum source 62 via a hose 66 (schematically depicted in FIG. 2).

The absorbent pad 24 is applied onto the skin-facing surface 52 of the drape 22 and is affixed to the drape 22 via the pressure-sensitive adhesive 50. Even though the absorbent pad 24 is shown to be rectangular in shape in plan view, the absorbent pad 24 could take other shapes so long as the absorbent pad 24 has a smaller area than the drape 22. As more clearly seen in FIG. 1, the absorbent pad 24 has a smaller area than the drape 22 so as to leave an area to affix the gasket 26 to the drape 22 surrounding the absorbent pad 24. The absorbent material from which the absorbent pad 24 is made can be a super absorbent polyester. Examples of such absorbent materials include a hydroactive wound pad available under the trademark Vilmed®. With reference to FIG. 2, a silicone coating 70 can be provided on a skin-contacting side of the absorbent pad 24, if desired, which is very compatible with skin and other tissue.

The gasket 26 can include silicone gel 76 applied on a silicone gel backing film 78. The silicone gel backing film 78 can be a polyurethane, polyethylene, polypropylene, or co-polyester film, that is brought in contact with the skin-facing surface 52 of the drape 22 to fix the gasket 26 to the drape 22. Typically, silicone does not bond well to an acrylic-based adhesive and the pressure-sensitive adhesive 50 is applied to the drape 22 in the illustrated embodiment. By providing the silicone gel 76 on the silicone gel backing film 78, the silicone gel 76 can be fixed with respect to the drape 22 while still being able to utilize a pressure-sensitive adhesive 50 on the drape 22. This allows for the benefit of providing a silicone gel 76 with the dressing 20 that can contact the skin around the tissue site and provide a much better seal than only having the pressure-sensitive adhesive 50, which allows for negative pressure to be obtained underneath the drape 22 around the wound site. The silicone gel 76 can operate as a sealing gasket for the dressing 20 to maintain negative pressure (with respect to atmosphere) underneath the dressing 20. Of the available biomedical materials, silicone gel is identified as a gasket candidate, such as the gel available from Polymer Science, Inc. as part number PS-1050. Other materials, such as hydrogel, could function as a sealing gasket but are not as biocompatible as silicone gel.

With reference back to FIG. 1, the gasket 26 has a frame shape in that an opening 82 is provided in the gasket 26 so that the gasket 26 surrounds the absorbent pad 24. The gasket 26 also includes an outer boundary edge 84 offset inwardly from an outer periphery 86 of the drape 22 leaving a border B of pressure-sensitive adhesive 50 around the gasket 26. This allows the border of pressure-sensitive adhesive 50 around the gasket 26 on the drape 22 to affix to the skin around the tissue site to hold the dressing 20 in place. Since pressure-sensitive acrylic-based adhesive is not particularly suitable for an airtight or watertight seal, the gasket 26 provides an adequate seal so that negative pressure can be maintained within a space beneath the drape 22 and surrounded by the gasket 26. Even though the gasket 26 is shown to be rectangular in shape in plan view, the gasket 26 could take other shapes.

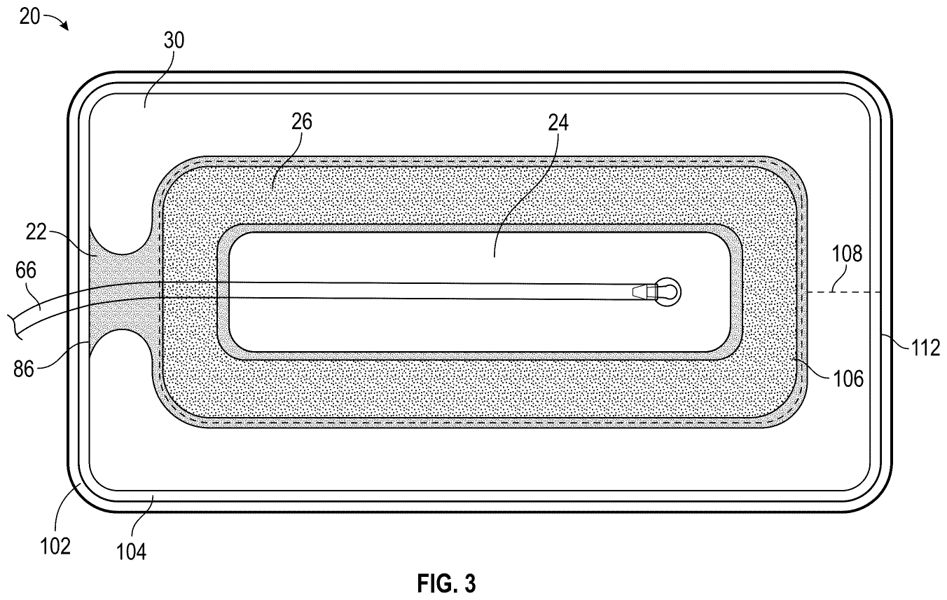

As mentioned above, the dressing 20 is provided with multiple release liners, which in the embodiment depicted in FIG. 3 include a gasket release liner 102 and a border release liner 104. With relatively larger dressings, for example, the dressing 20 in FIGS. 1-3 can have a longest dimension of about 16 inches (40 cm), placement of the dressing 20 on the tissue site can be unwieldy if the entire underside of the dressing 20 is exposed during initial placement of the dressing 20. Repositioning of the dressing 20 after the pressure-sensitive adhesive 50 has come in contact with the skin can be difficult; therefore, it can be desirable to allow for the dressing 20 to be positioned over the tissue site allowing the gasket 26 to retain the dressing 20 in place prior to adhering the dressing 20 to the skin using the pressure-sensitive adhesive 50.

As illustrated in FIG. 3, when assembled and prior to placement of the dressing 20 on the skin, the gasket release liner 102 is in contact with the gasket 26. In the embodiment depicted in FIG. 3, the gasket release liner 102 also covers the absorbent pad 24 when the gasket release liner 102 is in contact with the gasket 26. As discussed above, the silicone coating 70 can be provided on the skin-contacting side of the absorbent pad 24 which can help with maneuverability of the dressing 20 when placing the dressing 20 over the tissue site. With the dressing 20 depicted in FIG. 3, the gasket release liner 102 can be removed first while the border release liner 104 remains affixed to the drape 22. With the gasket release liner 102 removed from the drape 22, the gasket 26 can be appropriately positioned over the tissue site and because the gasket 26 includes the silicone gel 76, the dressing 20 can be more easily maneuvered with respect to the tissue site because much, if not all, of the pressure-sensitive adhesive 50 is still covered by the border release liner 104 that remains affixed to the drape 22. After the gasket 26 is appropriately placed over the tissue site, the silicone gel 76 can adhere to the skin even if not as well as a pressure- sensitive acrylic-based adhesive. Then, border release liner 104 can be removed exposing the border of pressure-sensitive adhesive 50 around the gasket 26 and the drape 22 can be affixed to the skin.

With reference to FIG. 3, both the gasket release liner 102 and the border release liner 104 are shown to be greater in area than the drape 22, but this need not be the case. In the embodiment depicted in FIG. 3, the gasket release liner 102 is placed beneath the border release liner 104 and each extend beyond the outer periphery 86 of the drape 22. When viewing from the underside of the dressing 20, the gasket release liner 102 can cover the border release liner 104. The gasket release liner 102 can be removed from the dressing 20 exposing the lower surface of the gasket 26 without removing the border release liner 104 from the border of pressure-sensitive adhesive around the gasket 26. The border release liner 104 is provided with a central hole defined by an inner edge 106 (shown in phantom in FIG. 3) that is offset laterally from the gasket 26. A cut line 108 can be provided from the inner edge 106 to an outer edge 112 of the border release liner 104 to facilitate removal of the border release liner 104 from the drape 22.

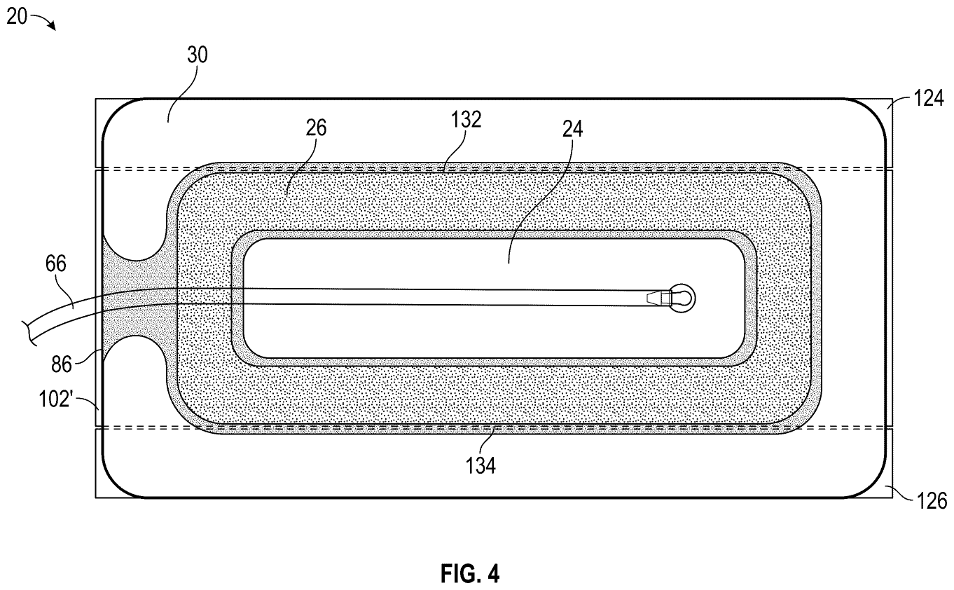

In the embodiment depicted in FIG. 4, the dressing 20 includes a gasket release liner 102′ that is not as wide as the gasket release liner 102 depicted in FIG. 3. As illustrated in FIG. 4, the gasket release liner 102′ is in contact with the gasket 26 and covers the absorbent pad 24 when the gasket release liner 102′ is in contact with the gasket 26. The dressing 20 also includes a first border release liner 124 and a second border release liner 126. In the embodiment depicted in FIG. 4, the first border release liner 124 is in contact with a portion of the border of pressure-sensitive adhesive around the gasket 26 and the second border release liner 126 is in contact with another portion of the border of pressure-sensitive adhesive around the gasket 26 of the drape 22. Both the first border release liner 124 and the second border release liner 126 in FIG. 4 are offset laterally from the gasket 26. In FIG. 4, the gasket release liner 102′ is also in contact with yet another portion of the border of pressure-sensitive adhesive around the gasket 26. Similar to the embodiment depicted in FIG. 3, in FIG. 4 the gasket release liner 102′ is removable from the gasket 26 to expose a lower surface of the gasket 26 without removing the first border release liner 124 or the second border release liner 126 from the pressure-sensitive adhesive 50.

In the embodiment depicted in FIG. 4, the gasket 26 is elongated in a direction parallel to a longest dimension of the drape 22. The first border release liner 124 covers a first portion of the border, which extends along a first relatively longer edge 132 of the gasket 26. The second border release liner 126 covers a second portion of the border, which extends along a second relatively longer edge 134 of the gasket 26. As seen in FIG. 4, the first relatively longer edge is opposite the second relatively longer edge.

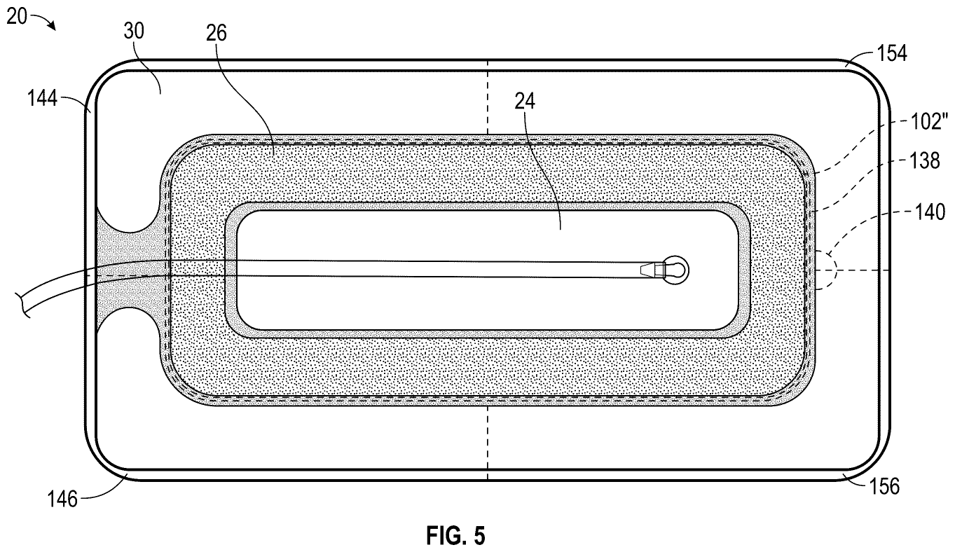

In the embodiment depicted in FIG. 5, the dressing 20 includes a gasket release liner 102″ that is not as wide as the gasket release liner 102 depicted in FIG. 3 and not as long as either the gasket release liner 102 depicted in FIG. 3 or the gasket release liner 102′ depicted in FIG. 4. As illustrated in FIG. 5, the gasket release liner 102″ is in contact with the gasket 26 and covers the absorbent pad 24 when the gasket release liner 102′ is in contact with the gasket 26. The gasket release liner 102″ in FIG. 5 has an outer peripheral edge 138 that substantially matches the shape of the outer boundary edge 84 (FIG. 1) of the gasket 26. The gasket release liner 102″ can also include a tab 140 extending from the outer peripheral edge 138 to facilitate removal of the gasket release liner 102″ from the gasket 26 and the absorbent pad 24.

In the embodiment depicted in FIG. 5, the dressing 20 includes a first border release liner 144 in contact with a first portion of the border of pressure-sensitive adhesive 50 around the gasket 26, a second border release liner 146 in contact with a second portion of the border of pressure-sensitive adhesive 50 around the gasket 26, a third border release liner 154 in contact with a third portion of the border of pressure-sensitive adhesive 50 around the gasket 26 and a fourth border release liner 156 in contact with a fourth portion of the border of pressure-sensitive adhesive 50 around the gasket 26.

In each of the embodiments depicted in FIGS. 1-5, the drape 22 is substantially rectangular. In the embodiment depicted in FIG. 5, the first border release liner 144 covers a first corner of the border, the second border release liner 146 covers a second corner of the border, the third border release liner 154 covers a third corner of the border, and the fourth border release liner 156 covers a fourth corner of the border. A longest dimension of the drape 22 defines a length. In the embodiment depicted in FIG. 3, the gasket release liner 102 and the border release liner 104 each extend along an entirety of the length of the drape 22. In the embodiment depicted in FIG. 4, the gasket release liner 102′, the first border release liner 124 and the second border release liner 126 each extend along an entirety of the length of the drape 22.

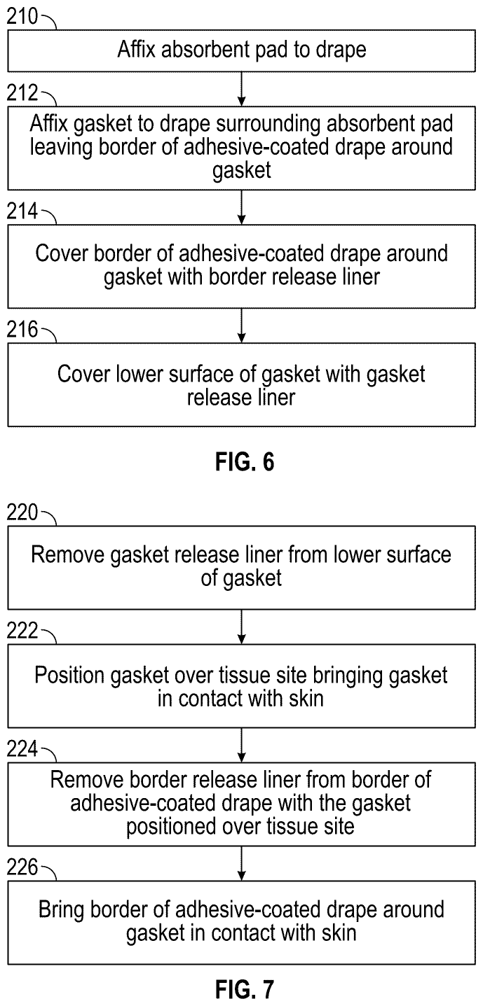

A method of assembling a dressing assembly will be described with reference to the flow diagram shown in FIG. 6 and the dressing assemblies shown in FIGS. 3-5. The method of assembly, however, is not limited to only the embodiment of the dressing 20 shown in FIGS. 1 and 2, nor is it limited to the particular order in which the steps are described or shown in FIG. 6, unless otherwise specifically mentioned.

At 210, the absorbent pad 24 is affixed to the drape 22. The skin-facing surface 52 of the drape 22 has the pressure-sensitive adhesive 50 deposited thereon. As such, the absorbent pad 24 sticks to the skin-facing surface 52 of the drape 22 leaving a margin of adhesive-coated drape around the absorbent pad 24.

At 212, the gasket 26 is affixed to the drape 22 surrounding the absorbent pad 24 leaving the border B of adhesive-coated drape around gasket 26. The silicone gel backing film 78 having the silicone gel 76 deposited thereon can applied onto the skin-facing surface 52 of the drape 22 so as to surround the absorbent pad 24 leaving the border B of adhesive-coated drape around gasket 26.

At 214, the border B of adhesive-coated drape around the gasket 26 is covered with the border release liner 104 in FIG. 3. The first portion of the border B of adhesive-coated drape around the gasket 26 can covered with the first border release liner 124 and the second portion of the border B of adhesive-coated drape around the gasket 26 can covered with the second border release liner 126 in FIG. 4. In FIG. 5, the first corner of the border B of adhesive-coated drape around the gasket 26 can be covered with the first border release liner 144, the second corner of the border B of adhesive-coated drape around the gasket 26 can be covered with the second border release liner 146, the third corner of the border B of adhesive-coated drape around the gasket 26 can be covered with the third border release liner 154, and the fourth corner of the border B of the adhesive-coated drape around the gasket 26 can be covered with the fourth border release liner 156.

At 216, the lower surface of gasket 26 is covered with gasket release liner 102 (FIG. 3), 102′ (FIGS. 4) and 102″ (FIG. 5). During this step, the skin-contacting side of the absorbent pad 24 can also be covered.

A method of applying the dressing 20 to skin will be described with reference to the flow diagram shown in FIG. 7 and the dressing assemblies shown in FIGS. 3-5. The method of application, however, is not limited to only the embodiment of the dressing 20 shown in FIGS. 1 and 2, nor is it limited to the particular order in which the steps are described or shown in FIG. 7, unless otherwise specifically mentioned.

At 220, the gasket release liner 102 (FIG. 3), 102′ (FIGS. 4) and 102″ (FIG. 5) is released from the lower surface of a gasket 26, which is affixed to the drape 22. Removing the gasket release liner 102 (FIG. 3), 102′ (FIGS. 4) and 102″ (FIG. 5) can further include uncovering the absorbent pad 24 that is affixed to the drape 22.

At 222, the gasket 26 is positioned over the tissue site and brought in contact with the skin. The gasket 26 can be appropriately positioned over the tissue site and because the gasket 26 includes the silicone gel 76, the dressing 20 can be more easily maneuvered with respect to the tissue site because much, if not all, of the pressure-sensitive adhesive 50 is still covered by the border release liner 104 that remains affixed to the drape 22.

At 224, the border release liner 104, in FIG. 3, is removed from the border B of adhesive-coated drape around the gasket 26 with the gasket 26 positioned over the tissue site. With reference to FIG. 4, the first border release liner 124 can be removed from the first portion of the border B of adhesive-coated drape around the gasket 26 and the second border release liner 126 can be removed from the second portion of the border B of adhesive-coated drape around the gasket 26. With reference to FIG. 5, the first border release liner 144 can be removed from the first corner of the border B of adhesive-coated drape around the gasket 26, the second border release liner 146 can be removed from the second corner of the border B of adhesive-coated drape around the gasket 26, the third border release liner 154 can be removed from the third corner of the border B of adhesive-coated drape around the gasket 26, the fourth border release liner 156 can be removed from he fourth corner of the border B of adhesive-coated drape around the gasket 26.

At 226, the border B of adhesive-coated drape around the gasket 26 can be brought in contact with the skin. At this time, the dressing 20 is appropriately affixed to the skin.

It will be appreciated that various of the above-disclosed embodiments and other features and functions, or alternatives or varieties thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. A dressing assembly comprising:
a drape formed of a flexible material capable of maintaining a negative pressure underneath the drape upon application of a vacuum;
pressure-sensitive adhesive provided on a skin-facing surface of the drape;
a gasket secured on the skin-facing surface of the drape, the gasket having a frame shape with an inner edge defining an opening and an outer edge offset inwardly from an outer periphery of the drape leaving a border of pressure-sensitive adhesive around the gasket;
a gasket release liner in contact with the gasket; and
a border release liner in contact with at least a portion of the border of pressure-sensitive adhesive around the gasket, the gasket release liner being removable from the gasket to expose a lower surface of the gasket without removing the border release liner from the border of pressure-sensitive adhesive around the gasket.

2. The dressing assembly of claim 1, further comprising an absorbent pad secured to the drape and surrounded by the gasket, wherein the gasket release liner covers the absorbent pad when in contact with the gasket.

3. The dressing assembly of claim 1, wherein the border release liner is a first border release liner in contact with a first portion of the border of pressure-sensitive adhesive around the gasket, and further comprising a second border release liner in contact with a second portion of the border of pressure-sensitive adhesive around the gasket.

4. The dressing assembly of claim 3, wherein the gasket is elongated in a direction parallel to a longest dimension of the drape, wherein the first border release liner covers the first portion of the border, which extends along a first relatively longer edge of the gasket, and the second border release liner covers the second portion of the border, which extends along a second relatively longer edge of the gasket, wherein the first relatively longer edge is opposite the second relatively longer edge.

5. The dressing assembly of claim 3, further comprising a third border release liner in contact with a third portion of the border of pressure-sensitive adhesive around the gasket and a fourth border release liner in contact with a fourth portion of the border of pressure-sensitive adhesive around the gasket.

6. The dressing assembly of claim 5, wherein the drape is substantially rectangular and the first border release liner covers a first corner of the border, the second border release liner covers a second corner of the border, the third border release liner covers a third corner of the border, and the fourth border release liner covers a fourth corner of the border.

7. The dressing assembly of claim 1, wherein the border release liner is offset laterally from the gasket so as not to cover the gasket.

8. The dressing assembly of claim 1, wherein a longest dimension of the drape defines a length, and each of the gasket release liner and the border release liner extend along an entirety of the length of the drape.

9. A method of assembling a dressing assembly comprising:
affixing an absorbent pad to a skin-facing surface of a drape;
affixing a gasket on the skin-facing surface of the drape surrounding the absorbent pad leaving a border of adhesive-coated drape around the gasket;
covering the border of adhesive-coated drape around the gasket with a border release liner; and
covering a lower surface of the gasket with a gasket release liner.

10. The method of assembling the dressing assembly of claim 9, wherein covering the lower surface of the gasket with the gasket release liner also includes covering the absorbent pad with the gasket release liner.

11. The method of assembling the dressing assembly of claim 9, wherein covering the border of adhesive-coated drape around the gasket with the border release liner includes covering a first portion of the border of adhesive-coated drape around the gasket with a first border release liner and covering a second portion of the border of adhesive-coated drape around the gasket with a second border release liner.

12. The method of assembling the dressing assembly of claim 9, wherein covering the border of adhesive-coated drape around the gasket with the border release liner includes covering a first corner of the border of adhesive-coated drape around the gasket with a first border release liner, covering a second corner of the border of adhesive-coated drape around the gasket with a second border release liner, covering a third corner of the border of adhesive-coated drape around the gasket with a third border release liner, covering a fourth corner of the border of adhesive-coated drape around the gasket with a fourth border release liner.

13. A method of applying a dressing to skin, the method comprising:
removing a gasket release liner from a lower surface of a gasket that is affixed to a drape;
positioning the gasket over a tissue site and bringing the gasket in contact with the skin;
removing a border release liner from a border of adhesive-coated drape around the gasket with the gasket positioned over the tissue site; and
bringing the border of adhesive-coated drape around the gasket in contact with the skin.

14. The method of claim 13, wherein removing the gasket release liner further includes uncovering an absorbent pad that is affixed to the drape.

15. The method of claim 13, wherein removing the border release liner includes removing a first border release liner from a first portion of the border of adhesive-coated drape around the gasket and removing a second border release liner from a second portion of the border of adhesive-coated drape around the gasket.

16. The method of claim 13, wherein removing the border release liner includes removing a first border release liner from a first corner of the border of adhesive-coated drape around the gasket, removing a second border release liner from a second corner of the border of adhesive-coated drape around the gasket, removing a third border release liner from a third corner of the border of adhesive-coated drape around the gasket, removing a fourth border release liner from a fourth corner of the border of adhesive-coated drape around the gasket.
